(19) United States
(12) Patent Application Publication
(10) Pub. No.: US 2011/0251084 A1
(43) Pub. Date: Oct. 13, 2011

(54) System for the Detection of a Biological Pathogen and Use Thereof

(76) Inventors: Colin Brenan, Marblehead, MA (US); Thomas Morrison, Winchester, MA (US)

(21) Appl. No.: 12/809,568

(22) PCT Filed: Dec. 22, 2008
(86) PCT No.: PCT/US08/14042

§ 371 Date: Jun. 28, 2011

Related U.S. Application Data
(60) Provisional application No. 60/015,555, filed on Apr. 2, 1996.

(51) Int. Cl. C40B 30/04; C12Q 1/68; C40B 60/08; C12M 1/40

(52) U.S. Cl. 506/9; 435/287.2; 435/6.15; 506/37

(57) ABSTRACT

The invention features compositions and methods that are useful for the detection of a target analyte, such as a pathogen, in a sample.

### CROSS-REFERENCE TO RELATED APPLICATION

This application claims the benefit of the following U.S. Provisional Application No.: 61/015,555, filed Dec. 20, 2007, the entire contents of which are incorporated herein by reference.

### BACKGROUND OF THE INVENTION

Following the anthrax attacks in late 2001, the medical and defense communities recognized the urgent need for accurate and sensitive methods for rapidly detecting biological pathogens that pose a health risk and/or a security risk. Despite rapid progress in polynucleotide and polypeptide detection methods, present methods for detecting pathogens are inadequate. There is an urgent need for methods that provide for the rapid identification and characterization of pathogens present in environmental or biological samples.

### SUMMARY OF THE INVENTION

As described below, the present invention features a system that provides for the rapid and sensitive detection of a target analyte, such as a human pathogen, in a sample, such as an environmental sample or a clinical specimen.

In one aspect, the invention provides a system for detecting a target analyte (e.g., a pathogen polynucleotide or polypeptide), the system containing a first module that provides for target specific amplification; a second module that contains a detector that detects the presence or absence of an analyte (e.g., one or more analytes, such as a polynucleotide and/or polypeptide); and a third module (that may be the same or different than the first module) containing means for target specific pre-amplification or amplification of a polynucleotide (e.g., a pathogen polynucleotide and/or polypeptide); and a detector that identifies the specific analyte. In one embodiment, detection of an analyte by the first module indicates that a target analyte is present in the sample and failure to detect an analyte indicates a target analyte is not present in the sample. In another embodiment, the system detects or identifies 1-50 or 1-20 (e.g., 1, 2, 3, 4, 5, 6, 7, 8, 9, 10, 11, 12, 13, 14, 15, 16, 17, 18, 19, 20, 25, 30, 35, 40, 45, 50) specific analytes. In another embodiment, target specific amplification occurs in a tube, bead, well, plate, channel, tubing, through-hole, microarray, or on a substrate. In yet another embodiment, the first module contains means for temperature control. In still another embodiment, the amplification is carried out in an array of through-holes. In still another embodiment, the detector detects a target specific polynucleotide and/or polypeptide. In another embodiment, the polynucleotide is bacterial, viral, fungal, or other pathogen. In another embodiment, the pathogen is any one or more of Vibrio cholerae, Legionella pneumonia, Cryptosporidium parvum, Staphylococcus aureus, and Bacillus anthracis. In another embodiment, the detector detects a Bacillus anthracis spore. In yet another embodiment, the pathogen is a plant pathogen that is any one or more of Myrothecium roridum, Phytophthora spp, Phytophthora infestans, Agrobacterium tum, Meloidogyne hapla, Colletotrichum coc, and Cylindocladium spatif, Verticillium dahlia, Verticillium albo-tric, Rhizoctonia solani AG 4-1, Rhizoctonia solani AG 2-2, G_proteob, Erwinea carot, Rhizoctonia solani AG 4-2, and Fusarium oxysporum. In still another embodiment, the first and third module are the same or different.

In a related aspect, the invention provides a method for detecting a pathogen, where the method employs a system delineated herein.

In another aspect, the invention provides a method for detecting and identifying a pathogen, the method involving amplifying a target specific polynucleotide in a sample; detecting the target specific polynucleotide, where detection of the polynucleotide indicates that the presence or absence of a pathogen in the sample; and identifying the target specific polynucleotide, thereby detecting and identifying the pathogen.

In another aspect, the invention provides a method for detecting and identifying a pathogen, the method involving amplifying a target specific polynucleotide in a sample; detecting the target specific polynucleotide, where the detection identifies the presence or absence of a target analyte in the sample; amplifying or pre-amplifying the target specific polynucleotide; and identifying the target specific polynucleotide, thereby detecting and identifying the pathogen.

In one embodiment, of any of the above aspects, the analyte, polynucleotide, or polypeptide is any one or more of Vibrio cholerae, Legionella pneumonia, Cryptosporidium parvum, Staphylococcus aureus, Bacillus anthracis, Myrothecium roridum, Phytophthora spp, Phytophthora infestans, Agrobacterium tum, Meloidogyne hapla, Colletotrichum coc, and Cylindocladium spatif, Verticillium dahlia, Verticillium albo-tric, Rhizoctonia solani AG 4-1, Rhizoctonia solani AG 2-2, G_proteob, Erwinea carot, Rhizoctonia solani AG 4-2, and Fusarium oxysporum. In still other embodiments, the method further involves detecting a pathogen polypeptide, for example, using an immunoassay. In various embodiments delineated herein, the sample is an environmental sample, such as an air sample, water sample, or environmental swab.

In another aspect, the invention provides a network of detection systems, the network containing at least 2, 3, 4, 5, 6, 7, 8, 9, 10, 11, 12, 13, 14, 15, 16, 17, 18, 19, 20, 25, 30, 35, 40, 45, 50 detection systems delineated herein in communication with a computer processing unit. Such systems are deployed within a building, such as a medical center, laboratory, hospital, airport terminal, military building, or governmental building. Alternatively, such systems are deployed within an area, such as a city, state, country, or area of military operations. In one embodiment, a detection systems delineated herein provides input regarding detection or identification of a pathogen to a computer processing unit, for example, via wireless communication method, ethernet connection, WiFi, mobile phone network, radio waves, blue tooth, microwave, or infrared methods.

In another aspect, the invention provides a combinatorial method for identifying multiple pathogens, the method comprising amplifying a target specific polynucleotide in a sample; and detecting the target specific polynucleotide using at least two probes each having a distinct detectable moiety, where detection of at least two moieties identifies the target specific polynucleotide, thereby detecting and identifying the pathogen. In one embodiment, the amplification is carried out in three separate PCR reactions and three dyes are used to detect at least 27 different targets.

In various embodiments of any of the above aspects, detection of an analyte by the first module or in a first stage indicates that a target analyte is present in the sample and failure to detect an analyte indicates a target analyte is not present in the sample. Where the system or method fails to detect an analyte, no further analysis of the sample is required. In another embodiment of an invention described herein, the system detects or identifies 1-50 or 1-20 (e.g., 1, 2, 3, 4, 5, 6, 7, 8, 9, 10, 11, 12, 13, 14, 15, 16, 17, 18, 19, 20, 25, 30, 35, 40, 45, 50) specific analytes (e.g., pathogen polynucleotides or polypeptides). In another embodiment of an invention described herein, target specific amplification occurs in a tube, bead, well, plate, channel, tubing, through-hole, microarray, or on a substrate. In yet another embodiment, the system includes means for temperature control. In still another embodiment, the amplification is carried out in an array of through-holes. In still another embodiment, the detector detects a target specific polynucleotide and/or polypeptide. In another embodiment, the polynucleotide is bacterial, viral, fungal, or other pathogen. In another embodiment, the pathogen is any one or more of Vibrio cholerae, Legionella pneumonia, Cryptosporidium parvum, Staphylococcus aureus, and Bacillus anthracis. In other embodiments, the detector detects a Bacillus anthracis spore. In still other embodiments, the pathogen is a plant pathogen that is any one or more of Myrothecium roridum, Phytophthora spp, Phytophthora infestans, Agrobacterium tum, Meloidogyne hapla, Colletotrichum coc, and Cylindocladium spatif, Verticillium dahlia, Verticillium albo-tric, Rhizoctonia solani AG 4-1, Rhizoctonia solani AG 2-2, G_proteob, Erwinea carot, Rhizoctonia solani AG 4-2, and Fusarium oxysporum. In still other embodiments, the first and third modules are the same or different.

Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “analyte” is meant any nucleic acid molecule, polypeptide, marker, or fragments thereof.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%.

By “amplify” is meant to increase the number of copies of a molecule. In one example, the polymerase chain reaction (PCR) is used to amplify nucleic acids.

By “binding” is meant having a physicochemical affinity for a molecule. Binding is measured by any of the methods of the invention, e.g., hybridization of a detectable nucleic acid probe, such as a TaqMan based probe, or Pleiades based probe.

By “detection system” is meant a set of one or more devices that provides for the detection and/or the identification of an analyte.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “sample” is meant any material collected for analysis.

By “detect” refers to identifying the presence, absence, or level of an analyte.

By “detector” is meant a device that distinguishes a signal.

By “detectable” is meant a moiety that renders an analyte detectable. Detection may be by any means known in the art, including but not limited to radiological, spectroscopic, photochemical, biochemical, immunochemical, or chemical means. Useful labels that render an analyte detectable include radioactive isotopes, magnetic beads, metallic beads, colloidal particles, fluorescent dyes, absorbing dyes, autofluorescent molecules, electron-dense reagents, enzymes, biotin, digoxigenin, haptens, aptamers, heavy metal atoms (substitute into DNA) and quantum dots.

By “detection of an analyte or pathogen” is meant identifying the presence or absence of an analyte or a pathogen in a sample.

By “identification of an analyte or pathogen” is meant identifying the

By “module” is meant a system component.

By “nucleic acid or oligonucleotide probe” is meant a polynucleotide capable of binding to a target nucleic acid of complementary sequence. Typically, such binding is accomplished through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

By “hybridize” is meant pair to form a double-stranded molecule between complementary polynucleotide sequences.

By “marker” is meant any protein or polynucleotide that is associated with a pathogen.

“Microarray” means a collection of nucleic acid molecules or polypeptides from one or more organisms arranged on a solid support (for example, a chip, plate, or bead). These nucleic acid molecules or polypeptides may be arranged in a grid where the location of each nucleic acid molecule or polypeptide remains fixed.

By “nucleic acid molecule” is meant an oligomer or polymer of ribonucleic acid or deoxyribonucleic acid, or analog thereof.

By “platen” is meant a device having a high-density array of holes for holding and/or analyzing a plurality of liquid samples, e.g., described in U.S. Pat. Nos. 6,716,629; 6,027,873; 6,306,578; or 6,436,632, all of which are herein incorporated by reference.

“Primer set” means a set of oligonucleotides that hybridize to one or more polynucleotides. A primer set would consist of at least 2, 4, 6, 8, 10, 12, 14, 16, 18, 20, 30, 40, 50, 60, 80, 100, 200, 250, 300, 400, 500, 600, or more primers.

By “reference” is meant a standard or control condition.

The phrase “selectively (or specifically) hybridizes to” refers to the binding, duplexing, or hybridizing of a molecule only to a particular nucleotide sequence under stringent hybridization conditions when that sequence is present in a complex mixture (for example, total cellular or library DNA or RNA).

By “specifically binds” is meant recognizes and binds a polypeptide or polynucleotide of the invention, but which does not substantially recognize and bind other molecules in a sample.

By “target nucleic acid molecule or polypeptide” is meant a nucleic acid molecule, polypeptide, or biomarker of the sample that is to be detected.

By “target specific amplification” is meant amplification of a target polynucleotide that occurs preferentially relative to non-specific amplification.

### BRIEF DESCRIPTION OF THE DRAWINGS

FIG. 1 is a schematic diagram of a system for detection of a target analyte.

FIG. 2 is a schematic diagram showing details for Stage 1 analysis.

FIG. 3 is a schematic diagram showing details of an alternative approach to Stage 1 analysis.

FIG. 4 is a schematic diagram showing direct detection of Stage 1 in detail, where a semi-quantitative dye scan is used during amplification.

FIG. 5 is a table showing how combinations of target specific primers and dye specific probes can be used for pathogen detection.

FIG. 6 is a table showing how combinations of target specific primers and dye specific probes can be used to detect specific pathogens.

FIG. 7 is a schematic diagram of a system for detection of a test analyte using multiple singleton PCR detection.

FIGS. 8A and 8B are a graph and a table showing that data obtained from Real-Time PCR on the OpenArray™ platform was highly reproducible. The standard deviation calculated across over 9000 data points was 0.11 Ct for 1000 target copies/through-hole, and 0.21 Ct for 100 copies/through-hole.

FIGS. 9A-9C show a work flow scheme for pathogen detection, the anticipated performance of the detection system, and the anticipated benefits of the detection methods, respectively.

FIGS. 10A and 10B includes three graphs and a table that provide a quantitative analysis of Vibrio parahaemolyticus, which was identified from a mix of 17 pathogens.

FIG. 11 includes four graphs showing detection of targeted regions of Vibrio cholerae, Legionella pneumonia, Cryptosporidium parvum and Staphylococcus aureus DNA samples. Real time PCR is performed using primer pairs for each of these four organisms. The X axis shows PCR cycle number. The Y axis shows fluorescence. NTC denotes no template control. This Figure is an example of a Stage 2 detection step following amplification. This figure shows that pathogen detection occurs in the open array. These graphs also shows the detection of specific amplification for each PCR product occurs in the presence of a mixture of pathogens.

FIG. 12 is a schematic diagram showing schematic PRI-lock principle for the detection of one target.

FIG. 13 is a schematic diagram showing an overview of the PRI-lock principle combined with the OpenArray technology for multiplex detection of three different targets,

FIG. 14 shows an image of real-time detection and quantification of a single target in serial dilutions within an OpenArray slide.

FIG. 15 shows real-time multiplex detection and quantification of 9 different plant pathogens in an OpenArray slide. Panel A shows the total number of used PRI-locks, targets in yellow were applied to the sample. Panel B shows the Spotting pattern of the through-hole subarray. Panel C shows a subarray picture after 32 cycles. Panel D shows Ct values for the found targets. The following pathogens were assayed: Myrothecium roridum, Phytophthora spp, Phytophthora infestans, Agrobacterium tum, Meloidogyne hapla, Colletotrichum coc, and Cylindocladium spatif, Verticillium dahlia, Verticillium albo-tric, Rhizoctonia solani AG 4-1, Rhizoctonia solani AG 2-2, G_proteob, Erwinea tarot, Rhizoctonia solani AG 4-2, and Fusarium oxysporum.

### DETAILED DESCRIPTION OF THE INVENTION

The invention features systems, methods and compositions for the detection and identification of a target analyte (e.g., polynucleotide, polypeptide) in a sample.

The invention is based, at least in part, on the discovery that a system for DNA extraction and concentration from a sample (e.g., environmental sample, air sample, water sample) may be coupled to a microfluidic system for the specific amplification and detection of one or more polynucleotides of interest (e.g., 1, 2, 3, 4, 5, 6, 7, 8, 9, 10, 11, 12, 13, 14, 15, 16, 17, 18, 19, 20, 30, 40, 50, 60, 75, 100). The system of the invention employs a two-stage strategy for identification of an analyte (e.g., identification of a pathogen polynucleotide). The first stage involves detection of the presence or absence of an analyte in a sample. This detection step is performed not to identify the analyte, but to determine if an analyte is present in the sample. The signal produced from the first detection step may be generated by the presence of one or more analytes in the sample. The second stage involves identification of the analyte. The system is particularly advantageous for the rapid low cost identification of even minute quantities of polynucleotide in a sample. In particular embodiments, the detection system provides for the identification of a pathogen polynucleotide or other analyte indicative of a biological threat.

The advantages of this two stage approach to target detection is multi-fold, particularly in the context of continuous monitoring of the environment for specific biological agents or for the rapid processing of a large number of clinical samples in the event of exposure to a contagious biological agent. In both cases, speed, accuracy and sensitivity of detection are key requirements for the detection system. The rapidity of detection system identification of a threat (i.e., the presence of a pathogen in a sample) will depend on the specific application, but generally identification of a pathogen in less than about two hours is desirable. Preferably, detection of the presence of a pathogen, and identification of the pathogen is accomplished in a time less than the physiological response to the infectious agent. Depending on the organism, this can be less than about 90 minutes, 60 minutes, or even 30 minutes. Sensitivity of the detection system to different micro-organisms has to be sufficient to detect an increase in the presence of the pathogen relative to the level of pathogens typically present in the environment. Desirably, the detection system provides for detection of a pathogen (e.g., micro-organism) at a level relative to the level of pathogen typically present in the environment. The present system advantageously provides for few false positive and false negative identifications, typically less than about 1:10⁵, 1:10⁶, 1:10⁷, 1:10⁸ to ensure the agent is reliably detected in the environmental sample or in the clinical specimen. In one embodiment, an internal positive control is included to decrease the false positive rate.

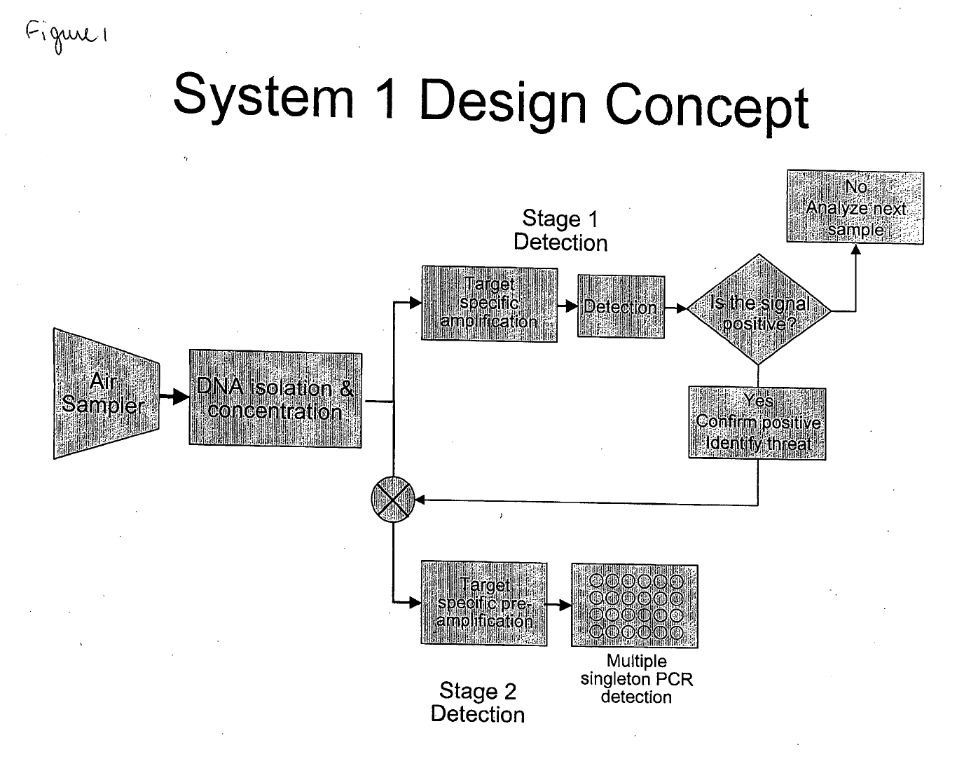

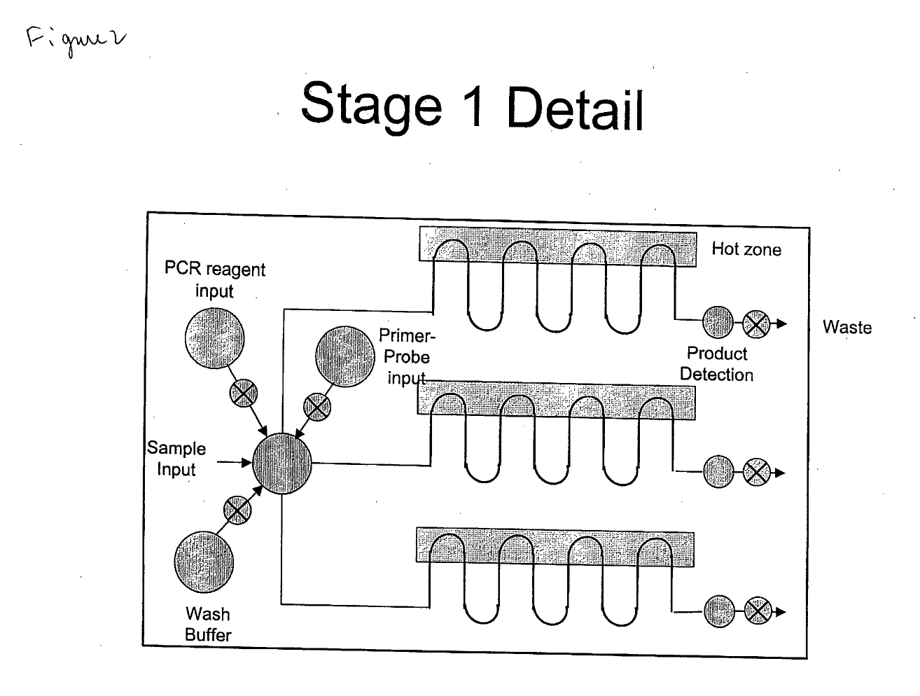

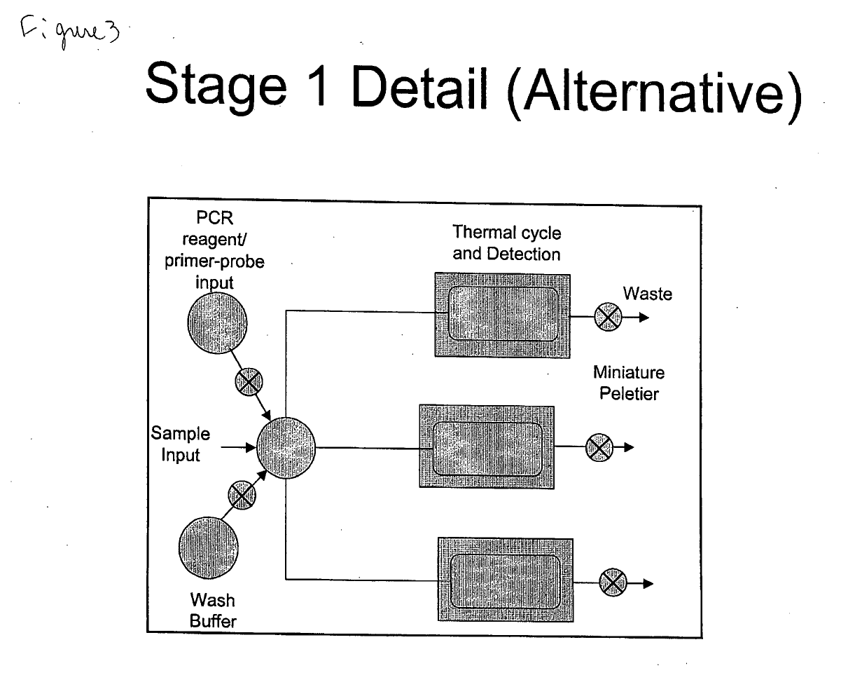

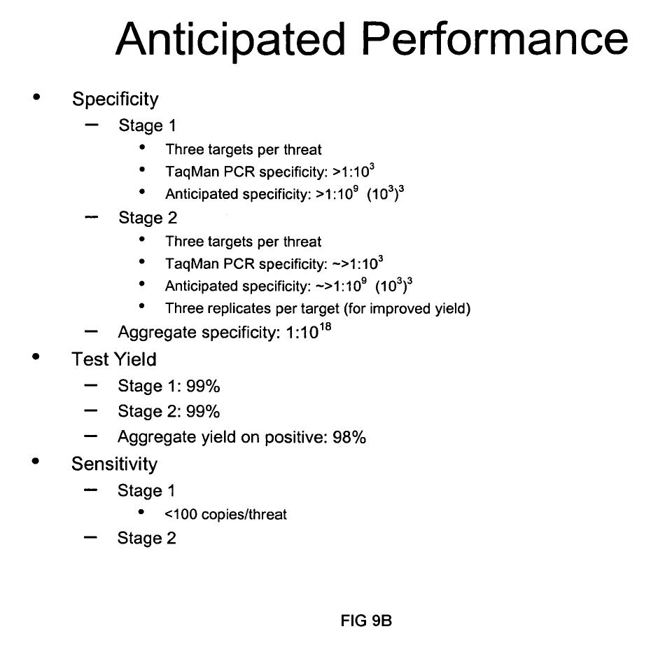

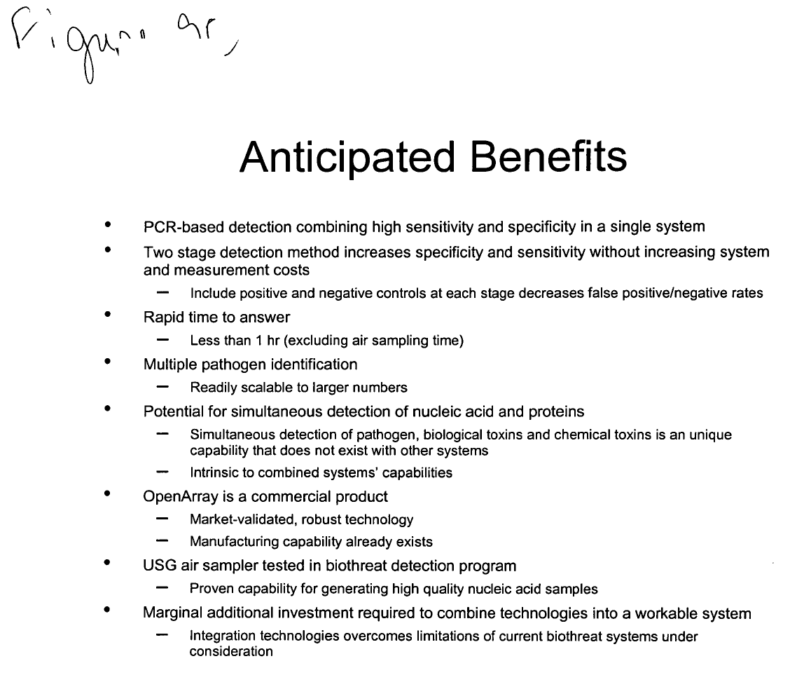

A detection system of the invention comprises a first component or Stage 1 that provides for target specific amplification (or pre-amplification) and a second component that comprises a detector (FIG. 1). A schematic diagram describing the work flow for pathogen detection is shown in FIG. 9A. The target specific amplification may be carried out in one or a series of tubes, wells, plates, channels, tubings, microarrays, through-holes or any other container or substrate that provides a suitable reaction environment. Preferably, the amplification is carried out in an array of tubes, wells, or through-holes. If desired, the first component further comprises means for temperature control or temperature cycling to facilitate an amplification reaction. For example, a temperature gradient may be imposed across one or two-dimensions of an array fabricated from thermally conductive material by holding at least one edge of the array at a specific temperature (e.g., creating a hot zone) (FIG. 2). If the temperature is changed with time, then the temperature distribution across the array can be varied. The rate of temperature change is generally proportional to the applied temperature and to the distance from the heat source. Temperature can be modified using any means known in the art, including but not limited to a peltier unit (FIG. 3), a water bath, ohmic heating of an electrically conductive plate or absorption of electromagnetic energy.

If the Stage 1 registers a positive result (i.e., indicates the presence of an analyte, such as a pathogen), the remaining sample is directed to the second stage for detection of the specific analyte(s) responsible for the positive signal observed in Stage 1. In Stage 2 the target may be pre-amplified using one or more pairs of target-specific primers, thereby generating one or more target-specific amplicons. The amplicons are then directed to one or more individual reaction containers (e.g., an array of microwells or through-holes) comprising one or more pairs of target specific primers and one or more probes. In one embodiment of Stage 2, at least one, two, three, four, or five targets (e.g., polynucleotide sequences) is assayed per pathogen. In other embodiments of Stage 2, at least about three or more replicates is assayed per threat. Internal positive and negative controls reduce false positive/negative rates in Stage 2.

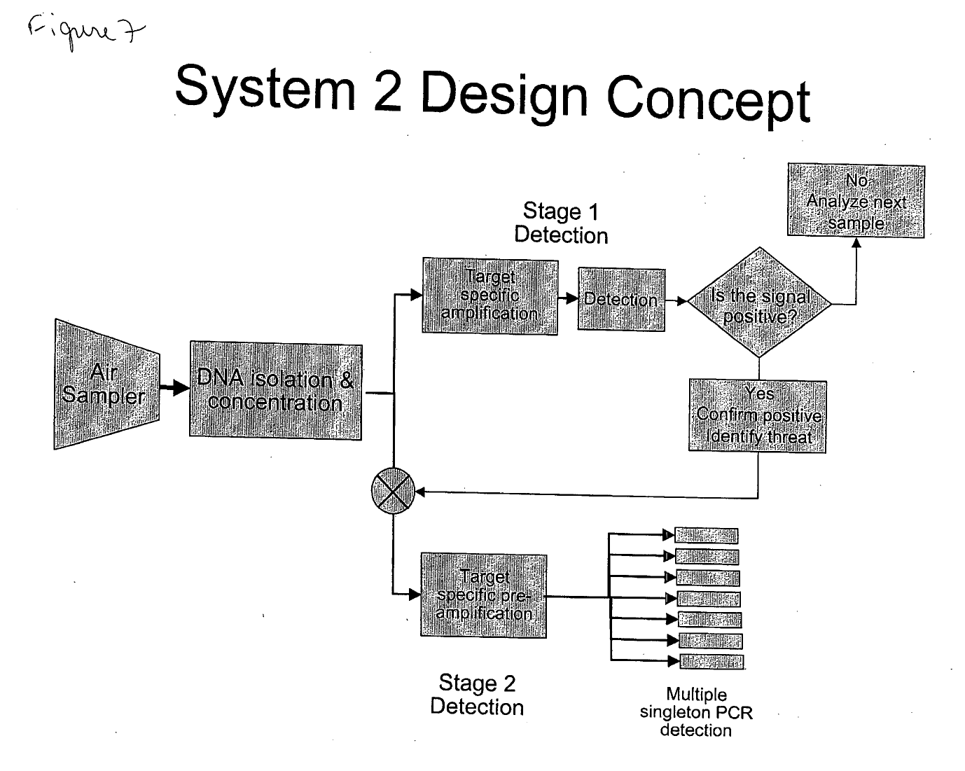

Alternatively, if a detectable amount of analyte is present following Stage 1, the analyte is identified, for example, by specifically binding a detectable probe or antibody. After the amplified sample is introduced into a container, the container is fluidically sealed and thermally cycled. The detection may be carried out in multiple singleton PCR detections (FIG. 7). If desired each reaction is monitored simultaneously in real-time for detection of an amplicon product for quantification of each target in the sample. Quantification is accomplished relative to a reference to internal controls. If desired, the array may include one or more of the following:
positive controls to establish that the assay(s) worked;
negative controls to identify the presence of a false positive result;
two, three, four or more replicates to increase the precision, yield and/or accuracy of the result; and,
multiple targets per pathogen may be detected to increase the specificity of the detection and to reduce the detection of false positives.
   In one embodiment, the sample from Stage 1 is divided into an array for detection of individual amplicons (FIG. 1). In another embodiment, the invention features a microfluidic circuit incorporating two detection stages, Stage 1 and Stage 2, where a test sample is split into two parts. One volume is directed for analysis in Stage 1 and if found to contain one or more analytes based on a positive signal, the second volume is directed for analysis in Stage 2 to identify the specific analytes responsible for the positive signal.

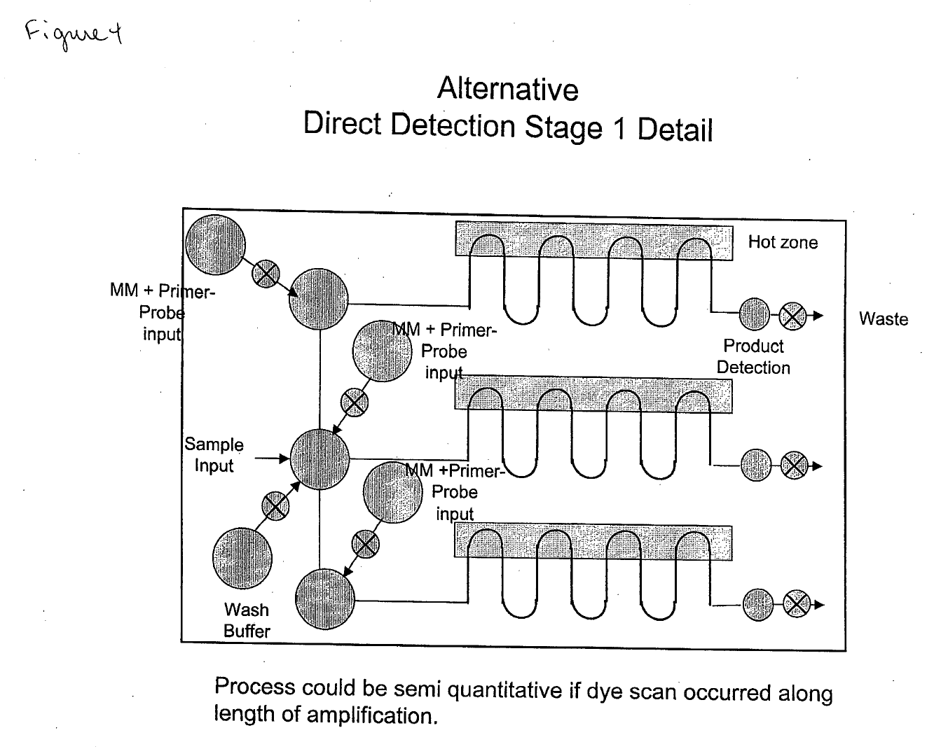

In order to speed up the PCR process, it is advisable to reduce the overall sample volume in order to reduce the thermal response time. As the reaction speed is determined by the time in which molecules find and bind to each other with the molecular transport being controlled by diffusion, the reduction of the diffusion length by reducing the size of the reaction vessel is advantageous. These objectives are achieved in continuous flow PCR format. The sample from Stage 1 is moved through a series of loops in a continuous PCR format that provides for heating and cooling of the sample as it traverses the loops and ultimately reaches the detector (FIG. 4). In this concept, the temperature cycling is achieved by transporting the sample in a microchannel through spatially fixed temperature zones. Only the moving liquid column has to undergo the temperature change between the PCR phases. Furthermore, this technique allows for a continuous flow of the process instead of the batch-processing of conventional thermocyclers. Desirably, the integration of an analysis step can also be achieved. In one embodiment, Stage 1 analysis is carried out using a continuous process.

Continuous-flow PCR chips may be manufactured in silicon, glass, disposable polymer microfluidic chips (e.g., polycarbonate) or metal. Such chips are microstructured using standard photolithography with a positive photoresist SU-8 and application of etching processes known in the art fabricate the microstructure pattern. Alternatively, the microstructure is derived by stamping the substrate with a stamp fabricated by subsequent electroplating of the developed resist structure with nickel. The channel dimensions may vary, however, in one embodiment the channel is 500 μm width, 100 μm depth with an overall channel length of 818 mm. After the inlet port, the liquid passes through a longer section of the microchannel at the denaturation temperature. The chip then contains some number (e.g., 5, 10, 15, 20, 30, 50 or more) temperature cycles that are effected by the passage of the sample through a channel that passes through one, two, three or more temperature zones before exiting the chip after a post-elongation period. The channel passes through a denaturing zone, an annealing zone and an elongation zone. The total length per PCR cycle is between 35 and 74 mm (e.g., 35, 40, 50, 60, 70, 80, 90, 100 mm), which represents a microliter sample volume that undergoes thermal cycling as compared to a liquid volume of 200 μl in a typical PCR plate. Desirably, the outer dimensions of the chip are 75.5 mm by 25.5 mm, the size of a microscopy slide for easy interface with standard laboratory equipment (Gartner et al., “Methods and instruments for continuous-flow PCR on a chip,” www.microfluidic-chipshop.com/files.php?dl_mg_id=33&file=dl_mg_{—}1199801133.pdf. In other embodiments, the continuous flow PCR is carried out on a disposable polymer disk having a long spiral microfluidic channel of varying width. The disc may be sandwiched with heat blocks of constant temperature (Chung et al., “Continuous-flow PCR using a disposable polymer disk (www-samlab.unine.ch/ConferenceCD/IMCS11/pdfs/AP190M.pdf). In one embodiment for continuous flow PCR, the sample is monitored by one or more detectors as it traverses the channel or channels. This provides for the quantification of the amplicon as it accumulates overtime.

Real-time PCR approaches are based upon a change in fluorescence associated with the accumulation of amplification products. The change in fluorescence is monitored in real time during thermal cycling. Fluorescence changes may be attributed to probe cleavage (e.g., TaqMan® chemistry), doublestranded DNA-binding dyes (e.g., SYBR® Green), primer extension (e.g., Molecular Beacons) or by incorporation of a fluorescence quencher to reduce the signal generated by a fluorescently-labeled primer (e.g., Plexor™ technology). PLEXOR™ Technology is used to detect product accumulation over time, which is measured as a reduction in fluorescent signal. Using the Plexor™ Systems, product accumulation is measured as a reduction in fluorescent signal during amplification. The reaction uses only two primers, one of which contains both a fluorescent tag and a modified base. The other primer is unmodified. As amplification proceeds, fluorescence is reduced by the site-specific incorporation of a fluorescent quencher, which is attached to a modified nucleotide (iso-dG) and inserted opposite the complementary modified base (iso-dC). The quencher is in close proximity to a fluorescent dye located on the 5′ end of the primer, resulting in a reduction in the fluorescent signal.

In yet another embodiment, the Stage 1 detects the presence of a pathogen using, for example, multiplex PCR. In Stage 2, one or more specific amplification products is used to identify the pathogen. In one embodiment, Stage 2 is carried out using batch processing for identification and quantification of positive threats in sample. To enhance specificity, two or more primer pairs may be used for each pathogen. Detection of each amplicon is then accomplished using specific fluorescent dyes. The detection of the specific combination of dyes indicates the positive identification of a pathogen in the sample. A positive control may be used to confirm that the assay worked. Failure of the assay could be associated with the presence of an inhibitor that interferes with PCR, fluidic error, reagent failure, or another problem.

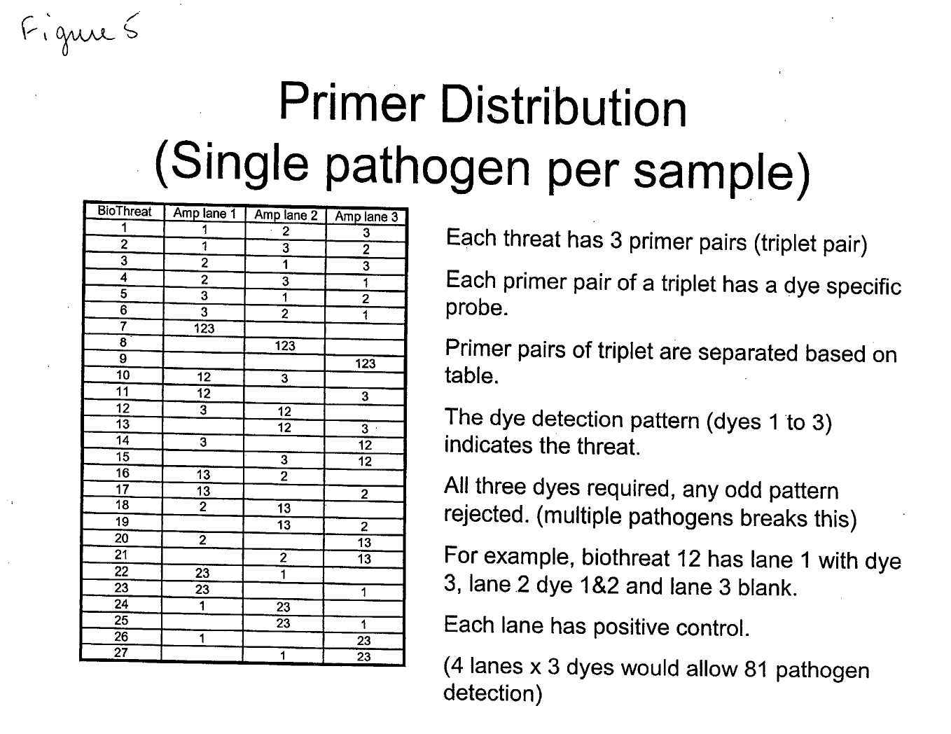

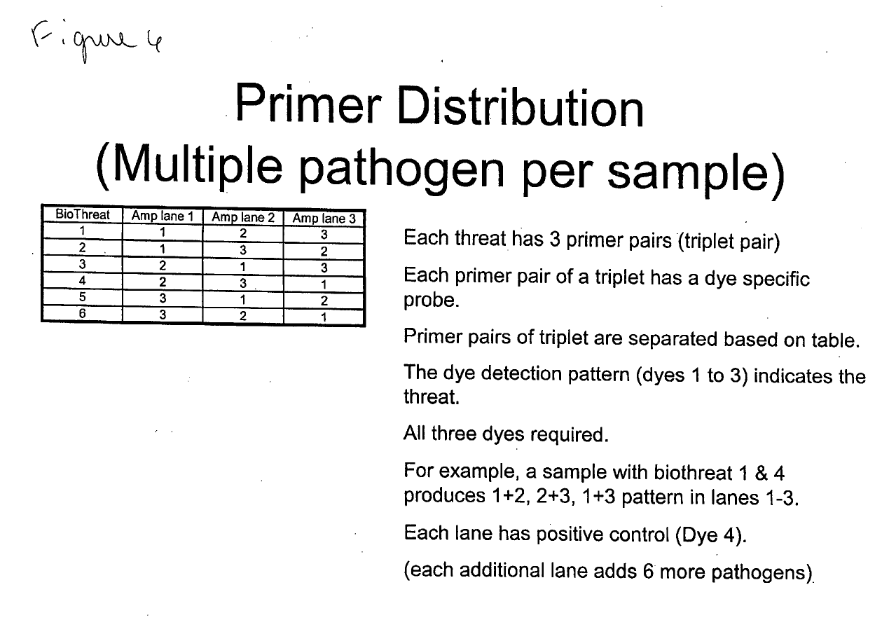

It may be desirable to monitor multiple pathogens directly in inexpensive microfluidic PCR. However, the number of pathogen identification per PCR reaction is limited by the detection methods employed in a thermal cycler reaction vessel. Melting curve analysis allows discrimination of PCR products based on the thermal melting properties of the PCR products and may be used to identify specific PCR products. Alternatively, fluorescent dyes are coupled to specific PCR events. These methods are commonly practiced and allow a single PCR reaction detect multiple pathogens. The number of pathogens identified by these detection methods is limited by the resolution of the detector. For example, the spectral separation of fluorescent dyes limits the number of specific probes used in a single reaction, or the thermal separation of melting products limits the number of uniquely identifiable PCR products. A simple solution is to run multiple PCR reactions, thereby multiplying the number of detectible targets by the number of additional PCR. The approach in FIGS. 5 and 6 uses a combinatorial approach that extends the number of detectable targets beyond a simple multiplicity. As shown in FIG. 5, three PCR and three dyes are used to detect up to 27 different targets. Each target would require amplification of three unique probes in a specific PCR reaction vessel before the target is considered present. Traditionally, an assay for 27 pathogens with three targets per pathogen would require 27 independent PCR using three different fluorescent dyes, instead of the three PCR reactions described by the approach depicted in FIG. 5. Advantageously, such methods use less sample than conventional methods. Although the Figures show three different PCR reactions each of which is detected in a separate microfluidic channel, one of skill in the art will appreciate that any number of combinatorial PCR reactions can be carried out. Advantageously, the present invention provides for multiple (2, 3, 4, 5, 6 or more) reactions to be carried out and analysed in one step. The sample is split into multiple separate reactions. If desired, the sample may be pre-amplified to generate enough sample to preserve sensitivity.

The approach described in FIG. 5 is particularly useful when the pathogen detected is rarely present, and multiple pathogens are unlikely to be identified in a single sample. FIG. 6 describes an alternative combinatorial approach that is particularly useful when multiple pathogens are likely to be present in a single sample. In this case three dyes and three independent PCR reactions resolve any combination of up to six pathogens (i.e., 1, 2, 3, 4, 5, 6). One of skill in the art appreciates that this method is readily adaptable to identify additional combinations of pathogens. This combinatorial approach can be incorporated with detection methods other than fluorescent dyes, such as melting curve profiles, capillary electrophoresis mobility, and charge to mass measured by mass spectrophotometer.

The output of the first stage may or may not be sufficient to unequivocally identify the pathogen. If sufficient amounts of polynucleotide are present, no additional amplification need be carried out. Instead, the polynucleotide from Stage 1 can be directly identified.

For example, if the first stage dye set is designed to signal the presence of bacteria, viruses or fungi in the specimen a different fluorescent probe is associated with the probe sequence targeting each class of micro-organism. Alternatively the same fluorescent probe or an intercalating dye such as SYBR Green I could be used with all primer sets and a generalized increase in fluorescent signal from the multiple reactions indicates the presence of a targeted micro-organism but does not identify the specific pathogenic agent.

In one embodiment, a reaction container comprises a plurality of reaction vessels (e.g., wells, through-holes) to accommodate the detection of a plurality of pathogens (e.g., greater than 1, 5, 10) simultaneously. The geometrical configuration of the array provides for rapid thermal cycling. In one embodiment, less than about one hundred twenty, sixty minutes, forty-five minutes, thirty minutes, twenty minutes, or fifteen minutes is required for detection of a positive or negative signal. In another embodiment, a positive or negative signal is detected in at least about 15 cycles, 20 cycles, 25, 26, 27, 28, 29, or 30 cycles. In another embodiment a positive or negative signal is detected in at least about 30, 35, 40, 45, 50, 55, or 60 cycles. If desired, nested primers are used in Stage 2 to increase the specificity of analyte detection and/or the specificity of PCR amplification.

The abundance of a target sequence is increased in the pre-amplification stage by PCR using target specific primers. The primers of the invention embrace oligonucleotides of sufficient length and appropriate sequence so as to provide specific initiation of polymerization on a significant number of nucleic acids in the target locus. Specifically, the term “primer” as used herein refers to a sequence comprising two or more nucleobases. Preferably, the primer comprises 3, and most preferably more than 8 nucleobases, which sequence is capable of initiating synthesis of a primer extension product, which is substantially complementary to a polymorphic locus strand. The primer must be sufficiently long to prime the synthesis of extension products in the presence of the inducing agent for polymerization. The exact length of primer will depend on many factors, including temperature, buffer, and nucleotide composition. The oligonucleotide primer typically contains between 12 and 27 or more nucleotides, although it may contain fewer nucleotides (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40). Primers of the invention are designed to be “substantially” complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with the 5′ and 3′ flanking sequences to hybridize therewith and permit amplification of the genomic locus.

Only a small number (e.g., 1, 3, 5, 10, 15, 20, 25, 30, 35, 40, 45, 50) of thermal cycles is needed to increase each target by over a thousand-fold above the background (e.g., 10 cycles=1024-fold increase). A small number of cycles in the presence of an abundance of primer means that the amplification remains in the exponential phase and therefore it is possible to achieve uniform and known amplification for each target sequence in the multiplexed reaction.

In an alternative embodiment the amplified mixture of amplicons from stage 1 could be introduced directly into stage 2 and dispensed in the multi-container structure for individual and discrete detection of specific DNA or RNA targets in each distinct reaction container.

The detector identifies the presence or absence of an analyte (e.g., pathogen polynucleotide) in the sample. The detector may distinguish any detectable signal in recognizing the analyte. A detector may employ spectroscopic, photochemical, biochemical, immunochemical, or chemical means to detect the presence of the analyte. Optionally, the detection system includes a processing unit that receives input from the detector regarding the presence or absence of the analyte.

In one embodiment, a PCR product (i.e., amplicon) or real-time PCR product is detected by probe binding. In one embodiment, probe binding generates a fluorescent signal, for example, by coupling a fluorogenic dye molecule and a quencher moiety to the same or different oligonucleotide substrates (e.g., TaqMan® (Applied Biosystems, Foster City, Calif., USA), Pleiades (Nanogen, Inc., Bothell, Wash., USA), Molecular Beacons (see, for example, Tyagi et al., Nature Biotechnology 14(3):303-8, 1996), Scorpions® (Molecular Probes Inc., Eugene, Oreg., USA)). In another example, a PCR product is detected by the binding of a fluorogenic dye that emits a fluorescent signal upon binding (e.g., SYBR® Green (Molecular Probes)). Such detection methods are useful for the detection of a target specific PCR product. Detection of the presence of a target triggers Stage 2, which provides for identification of the analyte.

In one embodiment, each reaction container comprises one or more pair of primers, each of which is complementary to a polynucleotide of interest, and capable of amplifying that sequence. If desired, the reaction container further comprises one or more probes that are detectably labeled. In one embodiment, the detectable probes each comprises a distinct fluorometric dye that provides for the separate detection of amplified target. In one embodiment, a microfluidic device or platen is in communication with an upstream sample collection device (e.g., an aerosol or liquid sampler). In another embodiment, the microfluidic device or platen is in communication with a downstream detector, i.e., an analytical device or means for carrying out analysis (e.g., mass spectroscopy (MS), nuclear magnetic resonance (NMR), capillary array electrophoresis (CAE), reverse transcription-PCR (RT-PCR), single molecule detection system, fluorescence detection, optical detection). Primer based fluorescent probes used for the primary detection step include but are not limited to LUX, which is commercially available from Invitrogen, and Plexor. LUX denotes Light-Upon-Extension, which refers to the probe's characteristic increase in fluorescent intensity upon incorporation in double stranded DNA.

Virtually any method known in the art may be used for detection of an analyte. If desired, one or more analytical methodologies can be performed on a through-hole, channel, reservoir, reaction chamber, capillary or combinations thereof.

The methods of the invention are particularly useful for monitoring for the presence of a pathogen in an environmental sample, such as an air sample, a water sample, an agricultural sample, or a biological sample. In one embodiment, the detection system described herein is coupled with a U.S. Genomics air sampling microfluidic system for DNA extraction and concentration to address the needs of the biodefense community. Specifications for the anticipated performance and benefits of the detection system described herein are listed in FIGS. 9B and 9C.

If desired, detection systems of the invention are deployed in a detection network. The network monitors for pathogen detection over an area. The network provides information on when a pathogen was first detected by a first detection system, whether the pathogen reached a second or third detection system, and the time that it took the pathogen to reach the next detector. This allows information to be gathered concerning, for example, the size, movement, and speed with which a pathogen or pathogen cloud is passing through an area. The network comprises detection systems that transmits information from a detection system to one or more central computers for processing.

Test analytes (e.g., polynucleotides, such as DNA, RNA) present in the sample are isolated and concentrated into a small volume, which is then split into two parts for a two-stage detection process. In the first stage, a preliminary determination is made that identifies the presence or absence of an analyte in the sample. The second stage provides for the identification of the analyte in the sample indicates the analyte. Detection of the presence of the target in the Stage 1 indicates that the sample should be further processed in a Stage 2 identification process. The Stage 2 increases the specificity and sensitivity of the assay, and definitively identifies the pathogen in the sample.

The present disclosure provides integrated modular systems for the preparation and analysis of target analytes from various samples. The systems are useful in the preparation and analysis of various target analytes, including but not limited to, molecules (e.g. toxins, such as Ricin, or pharmaceuticals), biomolecules (e.g., polynucleotides, polypeptides, lipids), cells (e.g., eukaryotic and prokaryotic cells, such as bacteria), spores (e.g., B. anthracis), viruses (e.g., influenza, smallpox,), and other materials. In various exemplary embodiments, microfluidic sample preparation and analysis can be performed by one or more of the system modules, as described herein.

In one embodiment, a first module for purifying, or concentrating a target analyte include one or more of the following methods, including but not limited to lysis, emulsification, sonication, centrifugation, chromatography, Solid Phase Extraction (SPE), immunocapture (e.g., immunomagnetic separations (IMS)), bead-based capture, and combinations thereof. In some embodiments, the first module can reduce a macroscale sample solution to a microscale volume, for example, by concentrating milliliters to microliters or smaller volumes for introduction into one or more microfluidic devices, such as a platen comprising a through-hole.

A “microfluidic device” as used herein refers to a device suitable for manipulating, storing, processing, or analyzing sub-milliliter quantities of fluid, such as microliter (μL), nanoliter (nL), and/or picoliter (pL) volumes. In various exemplary embodiments, a microfluidic device can comprise one or more microchips (e.g., micro-scale, nano-scale, pico-scale devices), capillaries or platens comprising through-holes.

### Microfluidic System

Advantageously, the invention employs a microfluidic or nanofluidic system that comprises a high density array of reaction containers, such as micro or nanoliter-scale through-holes, channels, or chambers for implementing a number of PCR analyses in less than about a 10, 20, or 100 microliters of fluid. In particular embodiments, the invention employs the BioTrove nanofluidic system—a high density array of nanoliter-scale through-holes or chambers for implementing up to 3072 PCR analyses with 33 nl per reaction on an array the size of a microscope slide. Such arrays are described, for example, by U.S. Pat. No. 6,716,629, which is incorporated herein by reference. The OpenArray (R) plate is a steel platen that comprises 3072 through holes having a diameter of about 320 μm. Each of the through holes is treated with a polymer to make the inside surface of each hole hydrophilic and the exterior surface hydrophobic. Liquid is dispensed and retained in each through-hole by means of surface force differentials between the liquid surface tension and the polymer coatings. The through holes are grouped in forty-eight subarrays of sixty-four through holes each. The spacing between each subarray is about 4.5 mm.

In particular, the invention provides a platen comprising a high density array of nanoliter-scale through-holes or chambers comprising less than about a 1000 nl, 750 nl, 500 nl, 250 nl, 100 nl, or even 50 nl of the reagents and samples for PCR analyses. Methods for loading the array with a small volume of reagents are described, for example, in U.S. Pat. Nos. 6,716,629, 6,812,030, and 6,716,629, and in U.S. Patent Publication Nos. 20080108112, 20030180807, and 20030124716. The hydrophobic exterior surface of the platen is not wetted, keeping the liquid in each through-hole isolated from its neighbor. The differential surface coating combined with the dimensional precision of the etched through-holes results in accurate and precise self-metered loading of liquid into each hole. PCR arrays are preloaded with PCR primers and probes. Such reagents are typically transferred from 384-well plates into the through-holes with an array of 48 pins manipulated by a 4-axis robot, such that each through-hole of an OpenArray (R) plate has a different primer set. The solvent is then removed resulting in the primers or primer/probes being immobilized on the inside surface of each hole. Co-loading of a passive fluorescent reference dye allows detection of holes that failed to load assay. The arrays are readily configurable as the assay configuration is based on the 384-well source plate layout. The 3072 holes of the OpenArray (R) plate may be configured based on analytical needs; for example a sample can be interrogated by 16, 32, 64, multiples of 64, up to 3072 assays.

The systems disclosed herein have widespread applications in biodefense monitoring, infectious disease diagnostics, forensics, genomics, proteomics and other fields. For biodefense, the technology provides compact units that may be deployed in the field to serve, for example, as pathogen monitoring devices for buildings, highways, cities, states, planes, airports, ships, or ports. The systems can prepare and analyze samples from air, biological fluids, agricultural products, or other sources to detect target pathogens. The combination of low consumable costs with automated preparation and analysis provides for a high throughput analytical system capable of screening a large number of samples simultaneously and identifying the presence or absence of a test analyte in the samples with high specificity. The systems disclosed herein also can be applied to pharmacogenetics, human medical genetics, biomedical research, animal and plant typing, and human identification.

Additional applications of the disclosed systems include molecular diagnostics, such as detecting microorganisms, genotyping organisms, sequencing, and forensics; creating sample preparation and analysis platforms for various methodologies, such as RT-PCR, sequencing, amino acid sequence detection, protein analysis, mass spectrometry, capillary array electrophoresis, differential display, and single molecule detection.

The two stage approach to detection of a target sequence in a specimen increases substantially the specificity of detection and greatly diminishes the false positives and false negatives of the measurement. Increased specificity comes about through the multiplicative combination of specificity for each detection stage. A signal is deemed a true positive based on observation of a positive signal in Stage 1 and a positive signal in Stage 2. To estimate this improvement, assume in both Stage 1 and Stage 2 the PCR assay amplifies three targets per micro-organism. The specificity of TaqMan PCR for each target is greater than 1:10³; thus for each target the anticipated specificity would be (10³)³=10⁹ as the output of the first stage (e.g., at least about 10⁷, 10⁸, 10⁹, 10¹⁰, 10¹¹). In one embodiment, the invention provides specificity associated with 10³ sensors×10⁴ measurements/sensor.

A similar specificity is observed for Stage 2 with a combined specificity of 10⁹×10⁹ or 10¹⁸ (e.g., at least about 10¹⁵, 10¹⁶, 10¹⁷, 10¹⁸, 10¹⁹, 10²⁰). A high specificity is important in the context of detection of biowarfare agents where a high false positive or false negative rate would make the detection system unreliable and unusable. With pre-amplification the sensitivity of the two stage system would be less than about 100 copies per micro-organism and up to at least 20 different micro-organisms per sample could be detected based on the multiplex-demultiplexed detection capability of the system. In particular, 200 copy×100 bp/copy/(1e⁻⁸ g/650 g/mole*6e23 bp/mole).

The two stage system design increases specificity and sensitivity without increasing system and measurement costs and combines high specificity and sensitivity in a single system. In one embodiment, the invention provides for the assay of at least about 10, 20, 50, 100, 150, 175, 200, 250, 300, 500, or even 1000 targets in a 5-10 ng DNA sample. In particular embodiments, the invention provides for the analysis of at least about 3, 5, 10, 20, 30, 40 or 50 threats (e.g., pathogens) in a single sample. In one embodiment, the samples are processed in batches or are processed continuously.

The time to answer is quick (less than about 1 hour, 2 hours, 3, hours) and the multiplexed initial detection combined with de-multiplexed, specific detection in the second stage enables detection of multiple pathogens simultaneously and is readily scalable to detect larger numbers of micro-organisms. The two stage system design intrinsically provides the high specificity and sensitivity needed for reliable and robust detection of a multitude of pathogenic organisms.

A variety of bacterial and viral pathogens may be detected using the system and methods of the invention. Exemplary bacterial pathogens include, but are not limited to, Aerobacter, Aeromonas, Acinetobacter, Actinomyces israelli, Agrobacterium, Bacillus, Bacillus anthracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, corynebacterium diphtheriae, corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Lactobacillus, Legionella, Leptospira, Listeria, Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia.

Examples of viruses detectable using the system and methods of the invention include Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

In particular embodiments, the invention provides systems and methods for the detection of pathogens that may be employed as biological weapons. Exemplary organisms that may be employed as biological weapons are provided in Table 1 (below).

TABLE 1

| | Traditional biological | Agents associated with |
|---|---|---|
| | warfare agents | biocrimes and bioterrorism |
| Pathogens | Bacillus anthracis^{b} | Ascaris suum |
| | Brucella suis | Bacillus anthracis^{b} |
| | Coxiella burnetii^{b} | Coxiella burnetii^{b} |
| | Francisella tularensis | Giardia lamblia |
| | Smallpox | HIV |
| | Viral encephalitides | Rickettsia prowazekii |
| | Viral hemorrhagic | (typhus) |
| | fevers^{b} | Salmonella Typhimurium |
| | Yersinia pestis^{b} | Salmonella typhi |
| | | Shigella species |
| | | Schistosoma species |
| | | Vibrio cholerae |
| | | Viral hemorrhagic |
| | | fevers (Ebola)^{b} |
| | | Yellow fever virus |
| | | Yersinia enterocolitica |
| | | Yersinia pestis^{b} |
| Toxins | Botulinum^{b} | Botulinum^{b} |
| | Ricin^{b} | Cholera endotoxin |
| | Staphylococcal | Diphtheria toxin |
| | enterotoxin B | Nicotine |
| | | Ricin^{b} |
| | | Snake toxin |
| | | Tetrodotoxin |
| Anti-crop agents | Rice blast | |
| | Rye stem rust | |
| | Wheat stem rust | |

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Screening and Validation

To test the specificity of a primer design strategy for pathogen detection and to examine the influence of multiple targets as well as various environmental water matrices on the quantitative capacity of Real-Time qPCR analysis on the OpenArray™ platform, DNA samples from 20 waterborne pathogens were interrogated against 110 assays (SYBR Green I primer pairs) designed to target agent's virulence and marker genes.

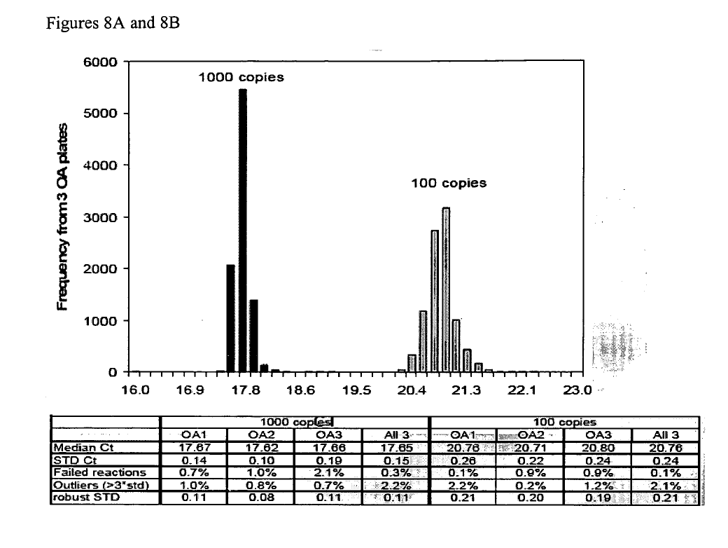

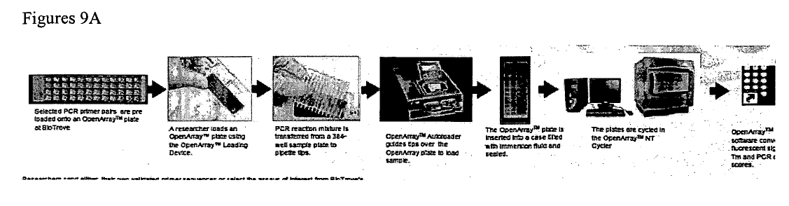

Real-Time PCR performance uniformity on the OpenArray™ platform was detected by conducting loading of 3072 through-holes with identical primer pair/sample combination. The experiment included three OpenArray™ plates cycled in parallel, resulting in 9216 data points. The data obtained from this experiment indicated highly reproducible Real-Time PCR on the OpenArray™ platform. The standard deviation calculated across over 9000 data points was 0.11 Ct for 1000 target copies/through-hole, and 0.21 Ct for 100 copies/through-hole (FIGS. 8A and 8B).

Validated primer sequences are loaded onto OpenArray™ plates. Standard PCR procedures, except the samples and master mix, are loaded onto OpenArray™ plates instead of other media. Researchers then seal the plates in glass cases, thermal cycle in the OpenArray™ NT Cycler. SYBR Green I fluorescence is collected and processed into cycle threshold (CT), amplicon melt temperature (Tm) and other PCR quality scores by the OpenArray™ NT Cycler Real-Time qPCR software.

Experiments were performed to test the specificity of a primer design strategy in a highly parallel manner. The primer pairs were designed to target up to 5 different regions per each studied pathogen. Assay sensitivity and specificity were determined by spiking serially diluted genomic DNA from individual organisms into DNA samples isolated from waste water, tertiary effluent and river water samples. Template DNA from 17 pathogens was tested individually with each set of designed primer pairs targeting up to five different regions of various pathogens. The mix of DNA isolated from 17 pathogens was spiked at different concentrations into background DNA that was 10-fold serially diluted, into the OA™ plates and cycled in the NT Cycler.

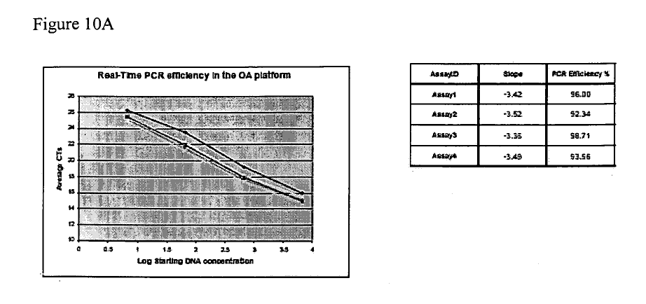

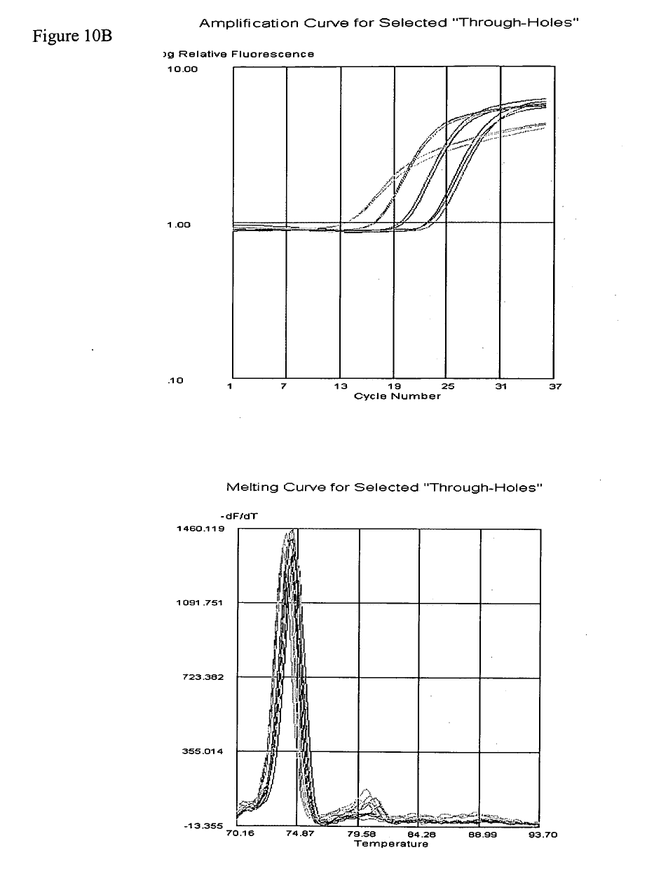

Quantitative analysis of Vibrio parahaemolyticus using assays designed to amplify four different regions of targeted pathogen and assay efficiencies were determined by measuring the slope of a plot of log starting DNA concentration against mean Ct values (FIG. 10A, shown in adjacent table). Observed differences in reaction efficiencies allow assay selection for further research. Vibrio parahaemolyticus was identified from a mix of 17 pathogens (FIG. 10B). The detection limit of the NT Cycler allowed the identification of as few as 6.6 genome equivalents of the pathogen. Also, spiking pure cultures in environmental samples had little influence on the quantitative capacity of the OpenArray™ system.

### Example 2

### Assay Sensitivity and Specificity

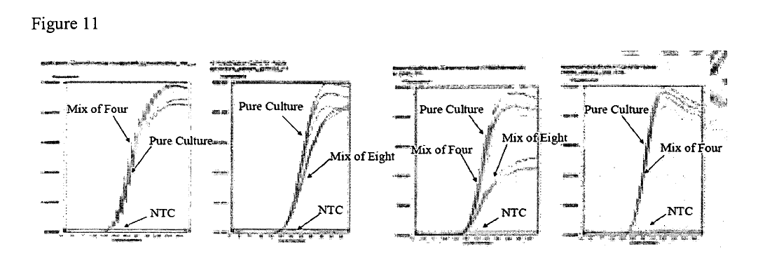

Assay sensitivity and specificity were determined by detecting genomic DNA from a single organism individually and in a mix of DNA from 4 or 8 organisms. Real-Time PCR performed on the OA™ platform allowed detection of targeted regions of Vibrio cholerae, Legionella pneumonia, Cryptosporidium parvum and Staphylococcus aureus DNA samples, isolated from pure culture as well as from a mixture of four or eight pathogens (FIG. 11).

Real-Time PCR on the OpenArray™ Platform is an ideal tool for highly parallel primer validation and multi-sample examination for pathogen detection. Advantageously data generated on the OA™ was highly efficient, reproducible and directly measured quantification via real time SYBR Green I Real-Time PCR. Assay design on the OA™ is quite flexible, enabling researchers to test unlimited variety of assay/sample combinations. Specificity of the assays is confirmed by dissociation curve analysis. Finally, the method is suitable for analysis of a variety of relatively low abundant microorganisms in high background DNA (i.e. environmental samples).

### Example 3

### Pri-Lock Probes and Real-Time PCR

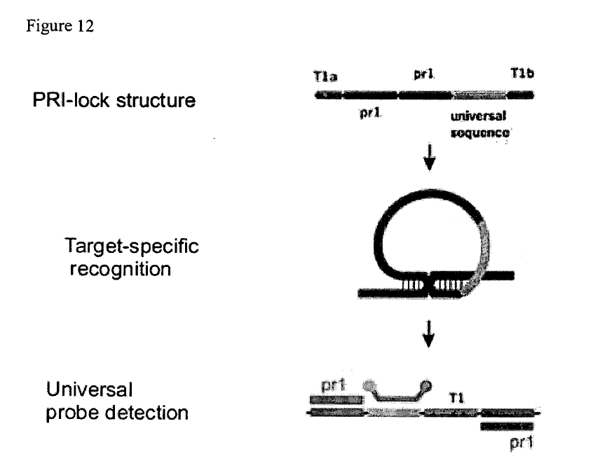

Recently, a conceptually new, ultra-high-throughput OpenArray™ platform has become available for real-time PCR, capable of accommodating more than 3000 reactions per array. Plant Research International has developed PRI-lock probes for multiplex detection which provide flexibility in target specific recognition and high-throughput amplification (Szemes et al., “Diagnostic application of Padlock Probes—Multiplex Detection of Plant Pathogens using universal microarray,” Nucleic Acids Research, 2005, Vol. 33, No. 8 e70). PRI-lock probes are long circularizable oligonucleotides with artificially selected unique primer pairs and an universal TaqMan probe region, flanked by target complementary regions (FIG. 12). In this study, the quantification power of circularizable ligation probes was characterized, and a highthroughput, quantitative multiplex diagnostic assay was developed.

### Example 4

### Multiplex Quantitative Target Detection

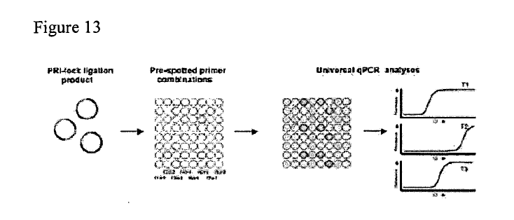

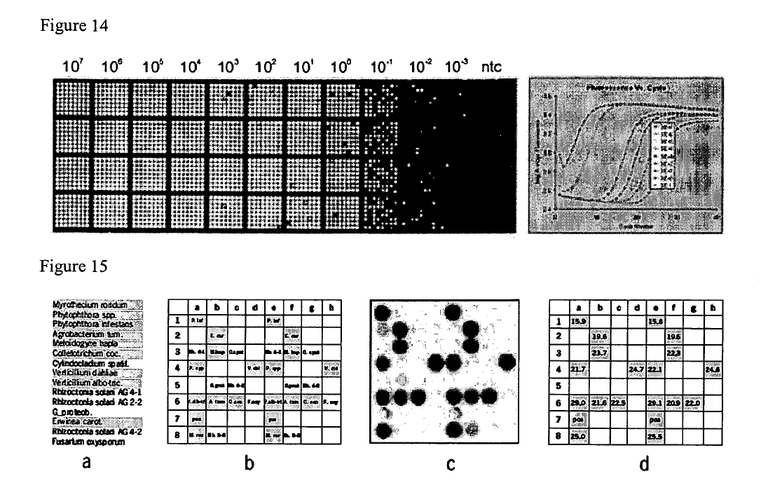

Real-time quantification for multiple targets is performed in an OpenArray. Multiplex PLP ligation is followed by single-plex amplification using unique primer pairs and an universal TaqMan probe in nano-liter PCR arrays (FIGS. 13, 14 and 15). Results shown in FIGS. 13, 14, and 15 show that PRI-lock/OpenArray technology provides high specificity, a high level of multiplexing, universal downstream processing after ligation. Importantly, this approach is flexible and easily adaptable. The high-throughput format was carried out with a universal TaqMan probe and quantitative multiplexing.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The following patents and patent applications may disclose related subject matter, and each is incorporated by reference in their entirety: 20070166743, 20050153354, 20050123974, 20040235014, 20040214211, 20040053399, 20030059822, 20010014850, U.S. Pat. Nos. 7,282,330, 6,927,065, 6,790,671, 6,772,070, 6,762,059, 6,696,022, 6,403,311, 6,355,420, 6,263,286, 6,210,896, 5,837,115, 5,427,663, 7,262,859, 6,927,065, 6,772,070, 6,762,059, 66,696, 6,263,286, as well as 20030180807 and 20030124716.

1-26. (canceled)

27. A system for detecting a target analyte, the system comprising:
a first module that provides for target specific amplification;
a second module that comprises a detector that detects the presence or absence of an analyte; and
a third module comprising means for target specific pre-amplification or amplification of a polynucleotide; and
a detector that identifies the specific analyte.

28. The system of claim 27, wherein detection of an analyte following amplification by the first module indicates that a target analyte is present in the sample and failure to detect an analyte indicates a target analyte is not present in the sample.

29. The system of claim 27, wherein target specific amplification occurs in a tube, bead, well, plate, channel, tubing, through-hole, micro array, array of through-holes, or on a substrate.

30. The system of claim 27, wherein said analyte is a Bacillus anthracis spore.

31. The system of claim 27, wherein the detector detects a target specific polynucleotide.

32. The system of claim 31, wherein said polynucleotide is bacterial, viral, fungal, or other pathogen.

33. The system of claim 32, wherein said pathogen polynucleotide is selected from the group consisting of Vibrio cholerae, Legionella pneumonia, Cryptosporidium parvum, Staphylococcus aureus, and Bacillus anthracis.

34. The system of claim 32, wherein said pathogen is a plant pathogen selected from the group consisting of Myrothecium roridum, Phytophthora spp, Phytophthora infestans, Agrobacterium tum, Meloidogyne hapla, Colletotrichum coc, and Cylindocladium spatif, Verticillium dahlia, Verticillium albo-tric, Rhizoctonia solani AG 4-1, Rhizoctonia solani AG 2-2, G_proteob, Erwinea carot, Rhizoctonia solani AG 4-2, and Fusarium oxysporum.

35. The system of claim 27, wherein the pathogen polypeptide is detected in an immunoassay.

36. The system of claims 27, wherein the sample is an air sample, water sample, or environmental swab.

37. A method for detecting a pathogen, wherein the method employs the system of claim 27.

38. A method for detecting and identifying a pathogen, said method comprising:
(a) amplifying a target specific polynucleotide in a sample;
(b) detecting the target specific polynucleotide, wherein said detection indicates the presence or absence of the polynucleotide in the sample, wherein failure to detect the polynucleotide indicates the pathogen is absent from the sample;
(d) optionally amplifying or pre-amplifying the target specific polynucleotide; and
(d) identifying the target specific polynucleotide.

39. The method of claim 38, wherein said polynucleotide is selected from the group consisting of Vibrio cholerae, Legionella pneumonia, Cryptosporidium parvum, Staphylococcus aureus, and Bacillus anthracis.

40. The method of claim 38, wherein said polynucleotide is selected from the group consisting of Myrothecium roridum, Phytophthora spp, Phytophthora infestans, Agrobacterium tum, Meloidogyne hapla, Colletotrichum coc, and Cylindocladium spatif, Verticillium dahlia, Verticillium albo-tric, Rhizoctonia solani AG 4-1, Rhizoctonia solani AG 2-2, G_proteob, Erwinea carot, Rhizoctonia solani AG 4-2, and Fusarium oxysporum.

41. The method of claim 38, wherein the method further comprises detecting a pathogen polypeptide, the pathogen polypeptide being optionally detected in an immunoassay.

42. A network of detection systems, the network comprising at least two detection systems according to claim 1 in communication with a computer processing unit.

43. The network of claim 42, wherein the detection systems provide input regarding detection or identification of a pathogen to the computer processing unit.

44. The network of claim 42, wherein the communication occurs via wireless communication method, ethernet connection, WiFi, mobile phone network, radio waves, blue tooth, microwave, or infrared methods.

45. A combinatorial method for identifying multiple pathogens, the method comprising
(a) amplifying a target specific polynucleotide in a sample; and
(b) detecting the target specific polynucleotide using at least two probes each having a distinct detectable moity, wherein detection of at least two moieties identifies the target specific polynucleotide, thereby detecting and identifying the pathogen.

46. The method of claim 45, wherein the amplification is carried out in three separate PCR reactions and three dyes are used to detect at least 27 different targets.
